(19) United States
(12) Patent Application Publication
(10) Pub. No.: US 2003/0235874 A1
(43) Pub. Date: Dec. 25, 2003

(54) Prostate-specific chimeric enhancers and methods of use thereof

(76) Inventors: Chinghai Kao, Zionsville, IN (US); Sang-Jin Lee, Indianapolis, IN (US); Hong-Sup Kim, ChungJu (KR); KangRyul Lee, Seoul (KR); Rong Yu, Chengdu (CN)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/431,791

(22) Filed: May 8, 2003

Related U.S. Application Data
(60) Provisional application No. 60/378,920, filed on May 8, 2002.

(51) Int. Cl. G01N 33/574; C07H 21/04; C12P 21/04; C12N 9/02; C12N 9/10; C12N 9/12; C12N 9/16; C12N 9/40; C12N 5/06

(52) U.S. Cl. 435/7.23; 435/69.7; 435/320.1; 435/189; 435/193; 435/194; 435/196; 435/208; 530/350; 536/23.2

(57) ABSTRACT

Compositions and methods are provided for the treatment of androgen-independent and androgen-dependent prostate carcinomas.

This invention claims priority under 35 U.S.C. §119(e) to U.S. Provisional Application No. 60/378,920 filed May 8, 2002. The entire disclosure of the above-identified applications is incorporated by reference herein.

Pursuant to 35 U.S.C. §202(c), it is acknowledged that the U.S. Government has certain rights in the invention described herein, which was made in part with funds from the National Institute of Health, Grant No. CA74042.

### FIELD OF THE INVENTION

This invention provides compositions and methods for the treatment of prostate cancer. More specifically, a novel prostate-specific chimeric promoter molecule is provided which may be used to advantage in innovative treatment modalities for androgen-independent and androgen-dependent prostate carcinomas.

### BACKGROUND OF THE INVENTION

All publications, patent applications, patents, and other references mentioned herein are incorporated by reference in their entirety. Full citations of the publications are found at the end of the specification.

The prostate is a walnut-sized gland located between the pubic bone and bladder. As men age, aberrant prostate growth is commonly observed. While benign prostate hyperplasia (BPH) is characterized by urinary tract obstruction due to prostate enlargement, malignant transformation of the prostate is accompanied by uncontrolled growth, invasion, metastasis and ultimately death.

Prostate cancer is the most commonly diagnosed cancer and the second leading cause of cancer death in men annually (1). Frequently, patients present with local advanced disease and/or detectable distant bone metastasis at initial diagnosis. The only treatment modality recommended for patients with advanced disease is androgen ablation therapy.

Tumor regression and improvement of clinical symptoms have been achieved by several means including castration, the use of diethylstilbestrol or luteinizing hormone releasing hormone agonist (LHRH) to lower circulating serum androgen, and by antagonizing androgen action with antiandrogens, such as, flutamide, cyproterone acetate or CASODEX®. However, the tumor regression observed with these treatment modalities is only temporary. Inevitably, the disease progresses to an androgen-independent state which is no longer effected by androgen ablation therapy. Unfortunately, there is no effective therapy available to treat androgen-independent prostate cancers.

Prostate specific antigen (PSA) and prostate specific membrane antigen (PSMA) are sensitive markers for prostate cancer diagnosis and progression. PSA is a serine protease produced by the prostatic ductal epithelial cells which is secreted into the seminal plasma. In normal prostate, a basement membrane acts as a barrier to block PSA from escaping into systemic circulation. However, in prostate cancer and benign hyperplasia, this membrane is disrupted and significant amounts of PSA are released into the blood stream.

PSA expression is modulated by androgen stimulation (2). When androgen binds to an androgen receptor (AR) on the cell surface, the primed AR is translocated into the nucleus and binds to the proximal promoter and enhancer core region (AREc) 4.2 kb upstream of the PSA gene to drive PSA expression (3-5). AREc harbors several androgen-response elements (AREs) and responds strongly to androgen induction (6). AREc also exhibits strong tissue-specific activity and has been manipulated to drive therapeutic gene expression targeting prostate cancer cells (7, 8).

Anti-androgen treatment of prostate cancer causes PSA serum levels in patients to drop precipitously (9, 10). However, more than 50% of all patients see a steady increase in PSA serum levels six months after surgical castration and/or anti-androgen therapy (11). This rebound in PSA serum levels indicates that the prostate cancer has become resistant to the anti-androgen treatment. Since PSA expression is controlled mainly by AR, it has been suggested that the rebound in PSA serum levels occurs because AR acquires functional activity in the absence of androgen.

Prostate specific membrane antigen (PSMA), a 100 kDa type II membrane glycoprotein with peptidase (12) and folate hydrolase activity (13), was originally identified as an antigen interacting with the prostate-specific monoclonal antibody, 7E11-C5 (14). The physiological role of PSMA in prostate remains unknown. Nevertheless, PSMA serum levels in prostate cancer are significantly higher than those observed in benign prostate hyperplasia or normal prostate, suggesting that enhanced expression of PSMA occurs during prostate cancer progression (15). Immunohistochemical staining, RT-PCR and in situ hybridization have demonstrated that PSMA is expressed predominantly in prostate tissue and tumor neovasculature. Expression of PMSA is elevated in bone and lymph node metastatic prostate cancers and in patients with recurrences after androgen deprivation therapy (16, 17).

Two characterized regulatory elements control PSMA expression. The proximal 1.2 kb promoter upstream from the PSMA encoding gene, FOLH1 , drives PSMA gene expression. The PMSA promoter demonstrates less tissue-specificity than AREc (18) as significant activation of the PSMA promoter has been demonstrated in several PSMA-negative cells (19, 20). The PSMA enhancer, PSME, was localized to the third intron of FOLH1. Unlike the 1.2 kb PSMA promoter, PSME promotes prostate-specific expression of PSMA (20). The tissue-specificity and enhanced up-regulation of gene expression in the absence of androgen mediated by this enhancer element prompted investigators to utilize this element in constructs for expressing toxic genes exclusively in the prostate in an androgen-depleted environment (21).

Because prostate cancers evolve to become androgen-independent and refractory to hormone ablation therapy, novel treatment modalities for prostate cancer must be developed that can effectively target androgen-independent and androgen-dependent prostate cancers.

### SUMMARY OF THE INVENTION

In accordance with the present invention, a novel prostate-specific promoter molecule, comprising prostate-specific enhancing sequence or PSES, has been developed which may be used to advantage in gene therapy approaches for the treatment of androgen-independent and androgen-dependent prostate carcinomas. PSES is a chimeric promoter molecule that comprises the promoter of PSA and the enhancer element of PSMA for driving gene expression in prostate cells. This novel chimeric promoter element is encoded by the nucleic acid sequence of SEQ ID NO: 1.

In one embodiment of the invention, the PSES promoter constructs are provided in which the PSES promoter is operably linked to a heterologous gene encoding a gene product, such as a reporter gene or a gene encoding a toxic protein, such that the PSES promoter controls the expression of the heterologous gene product. Suitable reporter genes for this purpose include, without limitation, luciferase, β-galactosidase, chloramphenicol acetyltransferase, secreted alkaline phosphatase (SEAP), and green fluorescent protein. In a particularly preferred embodiment of the invention, the heterologous gene encodes a toxic protein, e.g., cytosine deaminase, thymidine kinase or the death ligand, TRAIL (FIG. 5). In another preferred embodiment, the heterologous gene encodes an angiostatic gene, e.g., endostatin, tumstatin, sFLK-1 (KDR; kinase insert domain receptor), sFLT-1 (fms-like tyrosine kinase 1 receptor), TSP1 (thrombospondin), and sTie2 (TEK).

In yet another embodiment of the invention, expression vectors are provided which comprise a PSES promoter construct of the invention. In a preferred embodiment, the expression vector is an adenoviral vector containing the nucleic acid sequence of SEQ ID NO: 1 operably linked to a reporter gene or a gene encoding a toxic protein. Suitable adenoviral vectors include, without limitation, AdE1aPSESE1b, AdTK-PSESE1aPSESE1b, AdE1aPSESE1b-E4PSES-TK, AdAREc3E1aPSME(del2)E1b, AdAREc3E1aPSME(del2)E1b-E4PSES-TK, AdPSME(del2)E1aAREc3E1b, AdPSME(del2)E1aAREc3E1b-E4PSES-TK, AdTRAIL-PSESE1aPSESE1b, AdE1aPSESE1b-E4PSES-TRAIL, AdAREc3E1aPSME(del2)E1b-E4PSES-TRAIL and AdPSME(del2)E1aAREc3E1b-E4PSES-TRAIL. Isolated host cells are also provided which have been transformed with an expression vector of the invention in a pharmaceutically acceptable carrier.

In another embodiment of the invention, a method is provided for selectively targeting prostate carcinoma cells for destruction by administering to a patient a pharmaceutical composition containing a PSES promoter operably linked to a gene encoding a toxic protein. Alternatively, the pharmaceutical composition may comprise an adenoviral vector containing a PSES promoter construct of the invention.

In yet another embodiment of the invention, a method is provided for identifying test agents that modulate expression of the PSES promoter construct comprising the PSES promoter operably linked to a reporter gene. Test agents which bind to the PSES promoter construct are first identified and then PSES promoter activity is measured as a function of the reporter gene expression levels in the presence and absence of the test agents. The PSES promoter construct may also be introduced into a transgenic animal for identifying test agents which modulate expression of the PSES promoter construct as a function of reporter gene expression levels. In a preferred embodiment, the PSES promoter construct is used to generate a transgenic mouse and test agents are administered to the transgenic mouse. Suitable mice for this purpose include, without limitation, C4-2, CWR22rv and MDA Pca2b human prostate cancer model mice.

### BRIEF DESCRIPTION OF THE DRAWINGS

FIG. 1A shows the sequence of the 440 bp AREc (SEQ ID NO: 2) which is located 4.2 kb upstream of the PSA promoter. The linker-scanning mutations are underlined and the mutants are labeled L14 to R10.

FIG. 1B shows a graph illustrating the transcription activity levels of the 25 linker-scanning mutants within the AREc region. One ng of the Renilla luciferase vector, pRL-SV40 (Promega), was co-transfected to monitor transfection efficiency. Firefly and Renilla luciferase activities were determined using the Dual Luciferase Reporter System (Promega). Results are presented as mean±S.D. (standard deviation) of three transfections.

FIG. 1C shows a graph illustrating the transcription activity levels of the pGL3/AREc3/TATA, pGL3/p61/TATA, and pGL3/AREc/TATA constructs.

FIG. 1D shows the sequence of AREc3 (SEQ ID NO: 14). The linker-scanning mutations as shown in FIG. 1A are depicted. The AR and GATA binding sequences are also indicated.

FIG. 1E shows a graph illustrating the activity levels of AREc3 (C3) and AREc3L2m (C3L2m) in the presence or absence of androgen.

FIG. 1F shows the sequence of AREc3L2m (SEQ ID NO: 17). The linker-scanning mutations and AR and GATA binding sequences are depicted as shown in FIG. 1D.

FIG. 2A shows the PSME sequence (SEQ ID NO: 3) which is located in the third intron of FOLH1. PSME contains 3 AP-1 sites, a repeat sequence (marked by the box) and an Alu repeat sequence (heavily underlined).

FIG. 2B shows a schematic illustration (left panel) of the PSME deletions and a graph (right panel) illustrating the transcription activity levels of the promoter constructs containing the PSME deletions in the presence or absence of androgen (R1881). Results are presented as mean±S.D.

FIG. 2C shows the PSME(del2) sequence (SEQ ID NO: 18). AP-1 sites, a repeat sequence (marked by the box) and an Alu repeat sequence (heavily underlined) are marked as in FIG. 2A.

FIG. 3A shows a graph illustrating the transcription activity levels, in the presence or absence of androgen (R1881), of the PSES chimeric enhancer contructs: pGL3/PSME/TATA, pGL3/PSME/AREc/TATA, pGL3/AREc3/PSME, pGL3/PSME(del2)/TATA and pGL3/PSME(del2)/AREc/TATA.

FIG. 3B shows a graph illustrating the transcription activity levels of pGL3/PSES/TATA in various cell lines (2×10⁵ cells each) in the absence of androgen. Results are presented as mean±S.D.

FIG. 4A shows a schematic illustration of the construction of the recombinant adenovirus, Ad-PSES-luciferase.

FIG. 4B shows a graph illustrating the transcription activity levels of PSES in various cell lines (2×10⁵ cells each) infected by adenoviruses Ad-RSV-β-galactosidase and Ad-PSES-luciferase (100 virus particles per cell of each virus). Results are presented as mean±S.D.

FIG. 4C shows a graph illustrating the transcription activity levels of Ad-PSES-luciferase (empty bars) in various organs when injected into the tail vein of athymic mice (left panel) and directly into the prostate of male athymic mice (right panel). Filled bars are the activity levels with the control adenovirus Ad-CMV-luciferase.

FIG. 5 depicts a construct wherein a gene encoding a toxic protein is operably linked to AREc3 and PSME(del2). A particular operable linkage of AREc3 and PSME(del2) is also indicated by PSES. Other promoter and enhancer elements described herein can be interchanged with the those depicted (e.g., AREc3L2m (SEQ ID NO: 17 can replace AREc3).

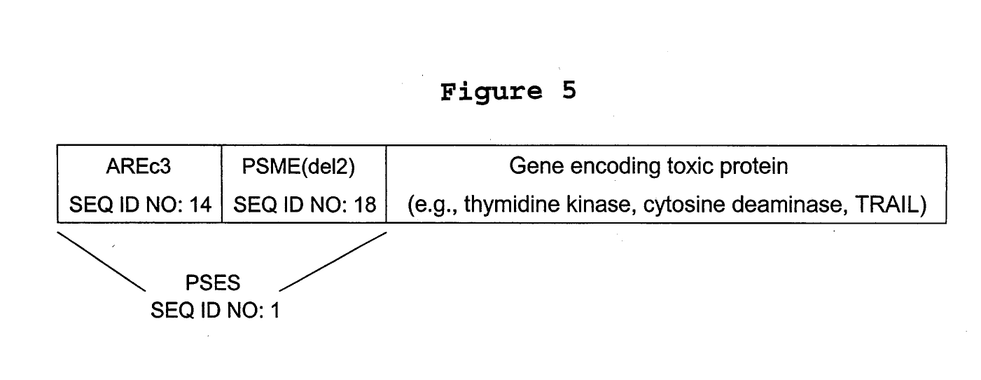

### DETAILED DESCRIPTION OF THE INVENTION

Androgens are essential for normal prostate development and influence the growth of prostate cancer cells. Currently, androgen ablation therapy is the only effective treatment modality for advanced prostate cancers. However, androgen ablation therapy is not effective against androgen-independent prostate cancer which ultimately leads to death.

In order to overcome the fatal results of androgen-independent prostate cancer (also known as hormonal refractory prostate cancer), a novel gene therapy strategy has been developed based upon the regulation of prostate specific antigen (PSA) and prostate specific membrane antigen (PSMA) promoter activities in androgen-independent prostate cancer cells. PSMA and PSA are well-characterized prostate epithelium-specific antigens, and the expression of these two marker proteins remains high in the hormone refractory stage of prostate cancer. An artificial chimeric promoter molecule, prostate-specific enhancing sequence or PSES, has been constructed from two modified regulatory elements that control the expression of the PSA and PSMA genes. This novel promoter remains silent in PSA/PSMA negative prostate and non-prostate cancer cell lines, but drives high levels of luciferase expression in PSA/PSMA-expressing prostate cancer cell lines in the presence and absence of androgen.

In one embodiment of the invention, the nucleic acid sequence encoding an exemplary PSES promoter molecule is disclosed. The nucleic acid molecule encoding the PSES promoter has the following nucleotide sequence:

(SEQ ID NO:1)

GATATTATCTTCATGATCTTGGATTGAAAACAGACCTACTCTGGAGGAAC
ATATTGTATCGATTGTCTTGACAGTAAACAAATCTGTTGTAAGAGACATT
ATCTTTATTATCTAGGACAGTAAGCAAGCCTGGATCTGAGAGAGATATCA
TCTTGCAAGGATGCCTGCTTTACAAACATCCTTGAAGCTAGCAATTATTT
TTTCCTTTAACCTTTCAAACTCAAGGAAAACCAGTTGGCCTTGACTCTGT
TTGTGGAAAATTTTAAACTACTGGTTTAATTTCTTTATTGGTTGTAATAT
GACTATTTTACGTCATATAACAATTTTTATTGTTTGTTAAATGACTTTAT
TGTTTGTCATATGATAATTTTATGTCATAGAACAATTTTTATTGCTTGAT
ATATGACTTTATTGTTATATGGCTATACAACTAGATTTTTTTGTTGTTTT
TGAC.

SEQ ID NO: 1 comprises SEQ ID NOs: 14 and 18 which are ARE and PSME elements, respectively. While SEQ ID NOs: 14 and 18 are contiguous in SEQ ID NO: 1, constructs where the ARE and PSME elements are separated by an intervening sequence yet still providing a synergistic enhancement of promoter function are encompassed within the scope of the instant invention.

In another embodiment of the invention, a recombinant adenovirus harboring the PSES chimeric promoter element encoded by SEQ ID NO: 1, Ad-PSES-luciferase, is provided. Luciferase activity in prostate cancer cell lines infected with Ad-PSES-luciferase is 400-1000 fold higher than in several other non-prostate cell lines thereby demonstrating the high tissue-specificity of the PSES promoter in an adenoviral vector. The luciferase activity from systemic injection of Ad-PSES-luciferase is fairly low in all major organs. However, when injected directly into the prostate, Ad-PSES-luciferase drives high levels of luciferase expression almost exclusively in prostate and not in other tissues.

The PSES promoter constructs of the invention may be used to advantage to treat both androgen-independent and androgen-dependent prostate carcinomas. Numerous applications employing of the PSES promoter of the invention for prostate cancer exist. For example, coding sequences for toxic enzymes including, but not limited to, cytosine deaminase (see, e.g., U.S. Pat. No. 6,552,005) and thymidine kinase (see, e.g., U.S. Pat. Nos. 6,555,108; 5,631,236; and 6,045,789), may be operably linked to the PSES promoter and incorporated into recombinant adenoviral vectors to kill prostate carcinoma cells regardless of androgen dependence. Alternatively, coding sequences for angiostatic proteins including, but not limited to, endostatin (GenBank accession number AF184060), tumstatin, sFLK-1 (KDR; kinase insert domain receptor), sFLT-1 (fms-like tyrosine kinase 1 receptor), TSP1 (thrombospondin), and sTie2 (ExTEK) may be operably linked to the PSES promoter and incorporated into recombinant adenoviral vectors to prevent angiogenesis within the tumor, regardless of androgen dependence (sequences of angiostatic agents available in pBlast series of plasmids from Invivogen, San Diego, Calif.).

Additionally, the PSES promoter construct may be used with replication-competent adenoviruses in a tissue-specific manner which are selectively cytolytic to prostate cancer cells. Suitable adenoviral vectors for this purpose include, without limitation, AdE1aPSESE1b, AdTK-PSESE1aPSESE1b, AdE1aPSESE1b-E4PSES-TK, AdAREc3E1aPSME(del2)E1b, AdAREc3E1aPSME(del2)E1b-E4PSES-TK, AdPSME(del2)E1aAREc3E1b, AdPSME(del2)E1aAREc3E1b-E4PSES-TK, AdTRAIL-PSESE1aPSESE1b, AdE1aPSESE1b-E4PSES-TRAIL, AdAREc3E1aPSME(del2)E1b-E4PSES-TRAIL and AdPSME(del2)E1aAREc3E1b-E4PSES-TRAIL. A more detailed description of these vectors is provided hereinbelow. These adenoviral vectors contain viral genes encoding E1A and E1B which are toxic to human cells. Transcription of these genes is controlled by the PSES, AREc and PSME promoters of the invention.

In yet another embodiment of the invention, the PSES promoter may be used to develop an animal model for studying prostate cancer. Previously, the PSA promoter was used to drive SV40 T-antigen expression in prostate to create a prostate cancer animal model in transgenic mice. PSES may also be used to advantage to generate transgenic mice for screening drugs that inhibit the detrimental actions of prostate-specific transcription factors.

The following description sets forth the general procedures involved in practicing the present invention. To the extent that specific materials are mentioned, it is merely for purposes of illustration and is not intended to limit the invention. Unless otherwise specified, general biochemical and molecular biological procedures, such as those set forth in Sambrook et al., Molecular Cloning, Cold Spring Harbor Laboratory (1989) (hereinafter “Sambrook et al.”) or Ausubel et al. (eds) Current Protocols in Molecular Biology, John Wiley & Sons (1997) (hereinafter “Ausubel et al.”) are used.

I. Definitions:

The following definitions are provided to facilitate an understanding of the present invention:

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

A “replicon” is any genetic element, for example, a plasmid, cosmid, bacmid, plastid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The phrase “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method of use. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transducer”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell or plant.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “percent similarity”, “percent identity” and “percent homology” when referring to a particular sequence are used as set forth in the University of Wisconsin GCG software program.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “functional” as used herein implies that the nucleic or amino acid sequence is functional for the recited assay or purpose.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, of that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by the trained artisan, and are contemplated to be within the scope of this definition.

The term “toxic protein” as used herein refers to proteins that, when synthesized in a transduced cell, induce cell death. Toxic proteins also include proteins that induce cell death by converting their non-toxic substrates called “prodrugs” into toxic substrates.

The term “angiostatic protein” as used herein refers to proteins that prevent the development of new blood vessels and/or inhibit new vessel growth.

A “clone” or “clonal cell population” is a population of cells derived from a single cell or common ancestor by mitosis.

A “cell line” is a clone of a primary cell or cell population that is capable of stable growth in vitro for many generations.

II. Preparation of Nucleic Acid Molecules:

Nucleic acid molecules of the invention may be prepared by two general methods: (1) synthesis from appropriate nucleotide triphosphates, or (2) isolation from biological sources. Both methods utilize protocols well known in the art. The availability of nucleotide sequence information, such as the DNA sequences encoding the prostate gene regulatory elements, enables preparation of an isolated nucleic acid molecule of the invention by oligonucleotide synthesis. Synthetic oligonucleotides may be prepared by the phosphoramidite method employed in the Applied Biosystems 38A DNA Synthesizer or similar devices. The resultant construct may be used directly or purified according to methods known in the art, such as high performance liquid chromatography (HPLC).

Specific probes for identifying such sequences as the PSA and PSMA regulatory elements may be between 15 and 40 nucleotides in length. For probes longer than those described above, the additional contiguous nucleotides are provided within SEQ ID NOS: 2 and 3.

Additionally, cDNA or genomic clones having homology with the PSA and PSMA genes may be isolated from other species using oligonucleotide probes corresponding to predetermined sequences within the PSA and PSMA nucleic acids of the invention. Such homologous sequences encoding PSA and PSMA may be identified by using hybridization and washing conditions of appropriate stringency. For example, hybridizations may be performed, according to the method of Sambrook et al., Molecular Cloning, Cold Spring Harbor Laboratory (1989), using a hybridization solution comprising: 5×SSC, 5× Denhardt's reagent, 1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

One common formula for calculating the stringency conditions required to achieve hybridization between nucleic acid molecules of a specified sequence homology (Sambrook et al., 1989) is as follows:

Tₘ=81.5° C.+16.6 Log [Na+]+0.41 (% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The nucleic acid molecules described herein include cDNA, genomic DNA, RNA, and fragments thereof which may be single- or double-stranded. Thus, nucleic acids are provided having sequences capable of hybridizing with at least one sequence of a nucleic acid sequence, such as selected segments of the sequence encoding PSA and PSMA. Also contemplated in the scope of the present invention are methods of use for oligonucleotide probes which specifically hybridize with the DNA from the sequences encoding PSA and PSMA under high stringency conditions. Primers capable of specifically amplifying the sequences encoding PSA and PSMA are also provided. As mentioned previously, such oligonucleotides are useful as primers for detecting, isolating and amplifying sequences encoding PSA and PSMA.

III. Methods of Drug Screening:

According to another aspect of the invention, methods of screening drugs for therapy, i.e., promoting or inhibiting PSES regulation of operably linked genes are provided.

The PSES promoter sequence elements employed in drug screening assays may either be free in solution, affixed to a solid support or within a cell.

An exemplary two step method entails identifying agents which bind to the chimeric promoter elements of the invention followed by biological assays wherein binding agents so identified are used in reporter gene assays to assess whether they modulate the activity of the PSES promoter as a function of reporter gene expression levels.

Reporter genes suitable for this purpose include, without limitation, β-galactosidase, luciferase, chloramphenicol acetyltransferase, and green fluorescent protein.

Methods for operably linking the coding regions for the reporter genes to the promoter sequence elements of the invention are well known to those of ordinary skill in the art.

Following introduction of such DNA constructs into recipient host cells, the cells may be contacted with agents suspected of affecting PSES promoter activity. Agents capable of altering expression of the reporter gene may prove efficacious in regulating PSES promoter activity, thereby having therapeutic advantage in the treatment of prostate cancer. For example, following introduction of a gene therapy vector comprising the PSES promoter operably linked to a sequence encoding a toxic protein into the prostate cells, such agents may be administered to the patient to increase the expression of the toxic protein thereby potentiating its effects.

Similarly, transgenic animals may be used to study the safety and efficacy of the agents suspected of affecting PSES promoter activity. For example, the PSES promoter operably linked to a gene encoding a toxic protein may be introduced into a prostate cancer animal model and agents suspected of enhancing PSES promoter activity may be assessed by monitoring the destruction of prostate cancer cells in the prostate cancer animal model. Suitable mouse models for this purpose include the C4-2, CWR22rv and MDA Pca2b human prostate cancer models.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to defined genetic loci, be randomly integrated within a chromosome, or it may be extra-chromosomally replicating DNA.

IV: Pharmaceutical Preparations:

The discovery that PSES promotes gene expression in androgen-independent prostate cancer cells facilitates the development of novel treatment methods and pharmaceutical compositions useful for the treatment of androgen refractory prostate carcinomas. Such pharmaceutical compositions may comprise, in addition to the PSES promoter operably linked to a sequence encoding a toxic protein, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, intramuscular or intraperitoneal routes.

Administration of the pharmaceutical preparation is preferably in a “prophylactically effective amount” or a “therapeutically effective amount” (as the case may be, although prophylaxis may be considered therapy), this being sufficient to show benefit to the individual.

Vectors, such as viral vectors, can be used to introduce PSES-toxic protein encoding nucleic acid constructs into a wide variety of different target cells. Typically the vectors are exposed to the target cells so that transformation can take place in a sufficient proportion of the cells to provide a useful therapeutic or prophylactic effect from the expression of the desired polypeptide. The transfected nucleic acid may be permanently incorporated into the genome of each of the targeted cells, providing long lasting effect, or alternatively the treatment may have to be repeated periodically.

A variety of vectors, both viral vectors and plasmid vectors are known in the art, see U.S. Pat. No. 5,252,479 and WO 93/07282. In particular, a number of viruses have been used as gene transfer vectors including, but not limited to papovaviruses including SV40, vaccinia virus, herpes viruses including HSV and EBV, and retroviruses. In a preferred embodiment of the present invention, adeno-associated viral vectors (see, e.g., U.S. Pat. No. 6,218,180) are used to introduce PSES controlled polynucleotide molecules to prostate carcinoma cells. In a particularly preferred embodiment of the present invention, adenovirus vectors are used to introduce PSES controlled polynucleotide molecules to prostate carcinoma cells. Notably, enhancers function in an orientation independent manner and therefore one enhancer can control the expression of two genes (31). Exemplary adenoviral vectors include without limitation: AdE1aPSESE1b (wherein the E1a and E1b adenoviral genes are under the control of a PSES enhancer), AdTK-PSESE1aPSESE1b (wherein a thymidine kinase (TK; such as herpes simplex virus TK) gene, E1a, and E1b genes are under the control of PSES enhancers), AdE1aPSESE1b-E4PSES-TK (wherein adenoviral genes E1a, E1b, and E4 and a TK gene are under the control PSES enhancers), AdAREc3E1aPSME(del2)E1b (wherein E1a is under the control of AREc3 and E1b is under the control of PSME(del2)), AdAREc3E1aPSME(del2)E1b-E4PSES-TK (same as AdAREc3E1aPSME(del2)E1b plus E4 and TK under the control of PSES), AdPSME(del2)E1aAREc3E1b (wherein E1a is under the control of PSME(del2) and E1b is under the control of AREc3), AdPSME(del2)E1aAREc3E1b-E4PSES-TK (same as AdPSME(del2)E1aAREc3E1b plus E4 and TK under the control of PSES), AdTRAIL-PSESE1aPSESE1b (wherein death ligand TRAIL (or soluble TRAIL; see e.g., U.S. Pat. No. 6,521,228; 30), E1a, and E1b are under the control of PSES enhancers), AdE1aPSESE1b-E4PSES-TRAIL (wherein E1a, E1b, E4, and TRAIL are under the control of PSES enhancers), AdAREc3E1aPSME(del2)E1b-E4PSES-TRAIL (wherein E1a is under the control of AREc3, E1b is under the control of PSME(del2), and TRAIL and E4 are under the control of PSES) and AdPSME(del2)E1aAREc3E1b-E4PSES-TRAIL (wherein E1a is under the control of PSME(del2), E1b is under the control of AREc3, and TRAIL and E4 are under the control of PSES). These viral vectors contain PSES, PSME and AREc controlled genes which are toxic to human cells.

The pharmaceutical preparation is formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In a particularly preferred embodiment of the invention, pharmaceutical preparations containing 10¹⁰ to 10¹³ pfu of adenoviral vector comprising the PSES promoter-toxic protein construct may be administered to a patient in need thereof. The appropriate dosage unit for the administration of pharmaceutical compositions comprising the PSES promoter-toxic protein construct may also be determined by evaluating the toxicity of the pharmaceutical compositions in animal models. Various concentrations of pharmaceutical preparations comprising the PSES promoter-toxic protein construct may be administered to mice with prostate cancer. The minimal and maximal dosages may then be determined based on the results of significant reduction in prostate tumor size as a result of the treatment. Suitable mouse models for this purpose include, without limitation, the C4-2, CWR22rv and MDA Pca2b human prostate cancer models (27-29). Moreover, the appropriate dosage unit may also be determined by assessing the efficacy of the treatment in combination with other standard prostate treatments, such as castration and androgen ablation therapy. Thus, the dosage units of pharmaceutical compositions comprising the PSES promoter-toxic protein construct may be determined individually or in combination with castration and/or androgen ablation therapy.

The pharmaceutical preparation comprising the PSES promoter-toxic protein construct may be administered at appropriate intervals (e.g. twice a day) until the prostate cancer tumors are reduced, after which the dosage may be reduced to a maintenance level. The appropriate interval in a particular case would normally depend on the condition of the patient. The route of administration of the constructs of the invention depends upon the condition of the patient. If the patient has low grade prostate cancer, the vectors of the invention are directly injected into the prostate. In patients having advanced disease, systemic administration via intravenous (i.v.) injection will be employed. Administration of the vector via this route should be effective to inhibit the growth of metasteses of prostate cancer in the bone.

While the above discussion refers to the delivery of pharmaceutical preparations comprising the PSES promoter-toxic protein and PSES-angiostatic protein constructs, it will be apparent to those skilled in the art that the methods described would also be suitable for the delivery of the vector constructs encoding PSES-toxic protein and PSES-angiostatic protein as well.

Further details regarding the practice of this invention are set forth in the following example, which is provided for illustrative purposes only and is in no way intended to limit the invention.

### EXAMPLE I

PSES Promoter Activity in Androgen-Independent Prostate Cancer Cells

A novel prostate-specific chimeric promoter, prostate-specific enhancing sequence or PSES, was constructed with the enhancer cores of the PSA and PSMA promoters, AREc3 and PSME(del2). PSES promoter activity was high in PSA/PSMA-positive prostate cancer cells in the presence and absence of androgens, and exhibited strong tissue specificity when inserted into an adenoviral vector.

I. Materials and Methods:

The following materials and methods are provided to facilitate the practice of the present invention:

Cells and Cell Culture. LNCaP, C4-2, CWR22rv, PC-3 and DU145 prostate cancer cell lines were maintained in T-media supplemented with 5% fetal bovine serum (FBS) and 1% penicillin/streptomycin (P/S). C4-2 was purchased from Urocore. CWR22rv was obtained from Dr. Liang Cheng. LNCaP was obtained from Dr. Leland Chung. The human liver carcinoma, HepG2; human cervix cancer cell line, HeLa; human testicular cancer cell line, Tera-1; human kidney cancer cell line, RCC-29; human colon cancer cell line, HT-29; human osteoblast-derived osteosarcoma cell line, MG63; and human prostate cancer cell lines, PC-3 and DU145, were purchased from American Type Culture Collection (Rockville, Md.) and maintained in DMEM supplemented with 5% FBS and 1% P/S.

Plasmid Construction. Full-length PSME was obtained by PCR-amplification using a BAC clone from Genome Systems (St. Louis, Mo.). Two primers, 5′-TTAGGCTAGCAATTATTTTTTCCTTTAACCTT-3′ (SEQ ID NO: 4) and 5′-ATCCCCCGGGAGGCGGAGGTTGCAGTGAGC-3′ (SEQ ID NO: 5), were used to amplify the PSME encoding nucleic acid and the fragments were digested with NheI/SmaI and ligated into pGL3/TATA (Promega, Madison, Wis.; 22).

PSME deletions were generated by PCR technology. Forward primers for PCR amplification were 5′-TTAGGCTAGCAATTATTTTTTCCTTTAACCTT-3′ (SEQ ID NO: 4) for PSME (del2), and 5′-TTAGGCTAGCCTTTATTGGTTGTAATATTGACT-3′ (SEQ ID NO: 6) for PSME (del3) and PSME (del4). The reverse primer was 5′-ATCCCCCGGGAGGCGGAGGTTGCAGTGAGC-3′ (SEQ ID NO: 5) for PSME (del3) and 5′-ATCCCCCGGGAAAACAACAAAAAAATCTAG-3′ (SEQ ID NO: 15) for PSME (del2) and PSME (del4). PCR-amplified fragments were digested with NheI/SmaI and ligated into pGL3/TATA.

The PSES promoter construct was constructed by digesting PSME(del2) with NheI/SmaI and inserting the resulting DNA fragment into pGL3/AREc3/TATA digested with NheI/SmaI.

PSA promoter derivatives were constructed as follows: AREc DNA fragments were prepared by PCR-amplification of p61 (22) using two primers, 5′-GGTACCCCTAGGGGTGACCAGAGCAGTCTA-3′ (SEQ ID NO: 7) and 5′-GCTAGCAGACAAGGGTGGAAGCCT-3′ (SEQ ID NO: 8), and subcloned into a TOPO cloning vector (Invitrogen, Carlsbad, Calif.). The resulting TOPO vector containing AREc was digested with KpnI/NheI and ligated into pGL3/TATA. pGL3/AREc3/TATA was then prepared by digesting PCR-amplified fragments with KpnI/NheI and inserting them into the pGL3/TATA vector. Amplification was performed using pGL3/AREc/TATA as a template and the two primers, 5′-GGTACCCCTAGGAGATATTATCTTCATGATC-3′ (SEQ ID NO: 9) and 5′-GCTAGCTTCAAGGATGTTTGTAAGC-3′ (SEQ ID NO: 10). All of the constructs used in this study were confirmed by sequence analysis.

Linker Scanning Mutagenesis. For linker scanning analysis of AREc, 25 linker mutants derived from pGL3/AREc/TATA were generated as described previously (22). These plasmids carry a mutant 440 bp AREc in which every 17 bases is replaced with a GAL4-binding site (CGGAGTACTGTCCTCCG; SEQ ID NO: 11). The forward primers and reverse primers contained half of the GAL4-binding site, GTCCTCCG (SEQ ID NO: 12) and AGTACTCCG (SEQ ID NO: 13) at the 5′-end, followed by 20 base pairs of AREc adjacent to the mutagenized bases. PCR reactions were carried out on a template of 100 pg pGL3/AREc/TATA. The PCR products were purified from 0.8% agarose gel and ligated with a rapid ligation kit (Roche, Indianapolis, IN). Clones were screened by PCR with a GAL4-binding site and Glprimer2 from the pGL3-basic vector (Promega). If a mutant demonstrated a 20% increase or decrease in promoter activity compared to AREc, it was subcloned back into pGL3/TATA to exclude the possibility that the effect was due to non-specific PCR mutation.

Transient Transfection for Luciferase and β-galactosidase Assays. 1×10⁵ cells/well were incubated in 12-well plates for 24 hours. Plasmid DNAs were introduced into the cells using DOTAP (Roche). Briefly, 0.5-1 μg of plasmid DNA was mixed with lipid at room temperature and after 15 minutes, the DNA-lipid complexes were added to a well containing 1 ml of serum-free and phenol red-free RPMI 1640 medium and incubated for 5 hours at 5% CO₂, 37° C. The DNA-lipid containing medium was then replaced with 1 ml of serum-free and phenol red-free RPMI 1640 medium containing 5% charcoal stripped serum and 1% P/S with or without 3 nM of androgen (R1881). After 2 days, cells were collected and lysed in 250 μl passive lysis buffer (Promega). Cell lysates were vortexed for a few seconds and centrifuged for 3 minutes.

For dual luciferase activity detection, the Dual-Luciferase™ Reporter Assay System was used (Promega). Briefly, 10 μl of the supernatant was mixed with 50 μl of luciferase substrate (Promega) and measured using a femtometer (Zylux, Germany). 50 μl of stop solution (Promega) was then added to the tube, mixed and measured using a luminometer to detect the renilla luciferase activity. For β-galactosidase activity detection, 20 μl of the supernatant was mixed with 200 μl of substrate (Tropix, Bedford, Mass.), incubated at 37° C. for 30 minutes and β-galactosidase activity was measured using a femtometer (Zylux). Data were expressed as relative luciferase activity, which was defined as luciferase activity normalized to internal control CMV/β-galactosidase activity for viral luciferase activity or SV40/renilla for dual luciferase activity. Relative luminescence was expressed as the mean±standard error of the mean of at least three independent experiments.

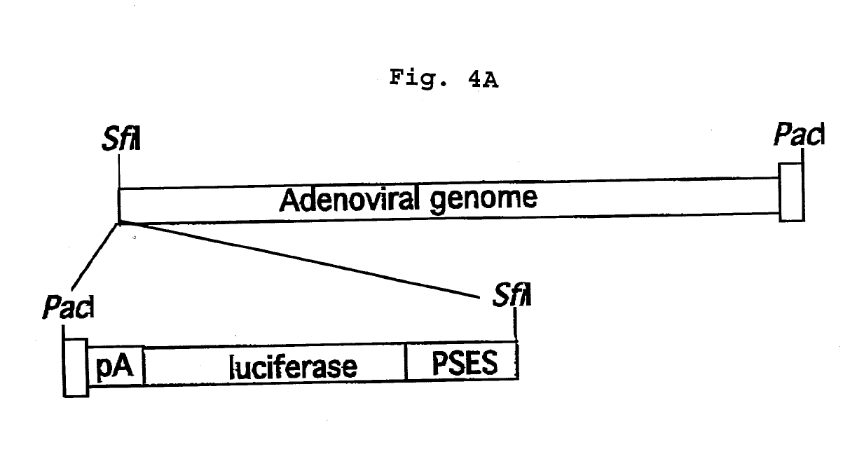

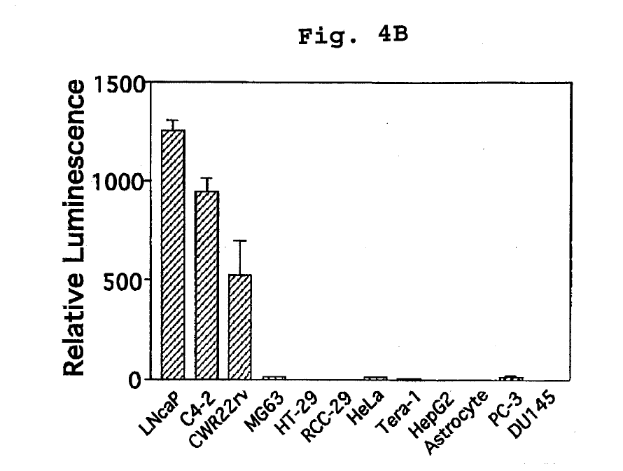

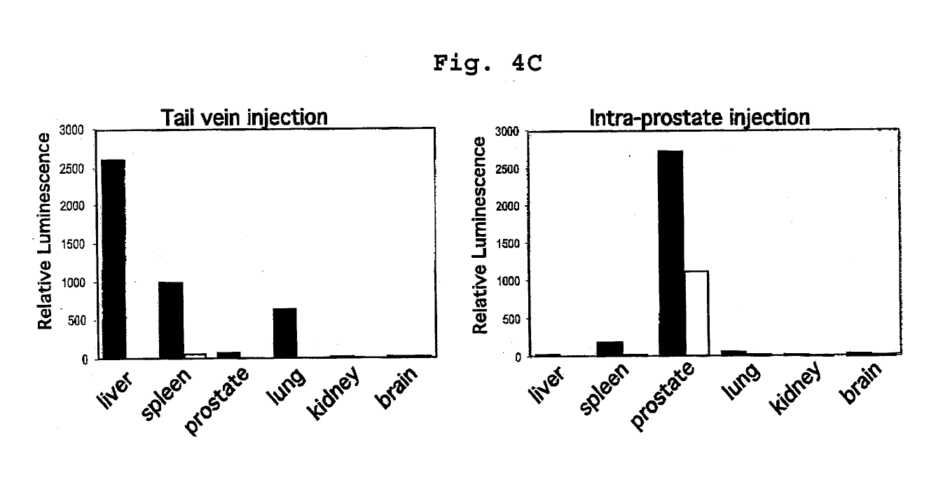

Virus Construction. The recombinant adenoviral vector containing PSES, called Ad-PSES-luciferase, was constructed as follows: A KpnI/BamHI fragment containing a PSES-luciferase expression cassette from pGL3/PSES/TATA was subcloned into pAd1020sfidA (a gift from Dr. Xavier Denthinne, OD 260 Inc, ID). pAd1020sfidA contains the adenovirus left ITR and packaging signal (base pairs 1 to 358). After cloning the expression cassette into pAd1020sfidA, the left arm of the adenovirus with the expression cassette was cut out by SfiI/PacI digestion and ligated to the SfiI/PacI adenovirus right arm, called AdenoZapsfi.2 (OD260), in the presence of PacI (FIG. 4A). The ligation mixture was precipitated and transfected into 293 cells using lipopectamine (GibCo, Rockville, Md.). The cells were passed from 24-well to 6-well growth conditions and covered with 0.6% agarose 2 days after transfection. Several selected clones were obtained after at least three rounds of plaque isolation using methods described previously (23). One clone was propagated in 293 cells, purified by CsCl₂ gradient centrifugation and dialyzed against 10 mM Tris-HCl (pH 7.5)/1 mM MgCl₂buffer supplemented with 10% glycerol. The viral titer was determined by measuring the optical density at 260 nm after lysing viral particles in 5% SDS. The control viruses, Ad-RSV-β-galactosidase and Ad-CMV-luciferase, were prepared as described elsewhere (23).

In Vivo Animal Experiments. Ba1b/c nude mice (athymic mice) were injected through the tail vein with 7.5×10¹ virus particles of Ad-CMV-luciferase or Ad-PSES-luciferase. For intra-prostatic injection, 7.5×10⁹ virus particles of Ad-CMV-luciferase or Ad-PSES-luciferase were injected directly into the prostate. Animals were sacrificed and their major organs were collected. Three days after virus injection, organs in 1 ml of 1×passive lysis buffer (Promega) supplemented with protease inhibitor cocktails were homogenized using a PowerGen 125 (Polytron Kinematica, Switzerland). 50 μl of lysate was then analyzed for luciferase activity as described above.

II. Results and Discussion:

AREc3 and PSME (del2) Exhibit Strong Prostate-Specific Activity.

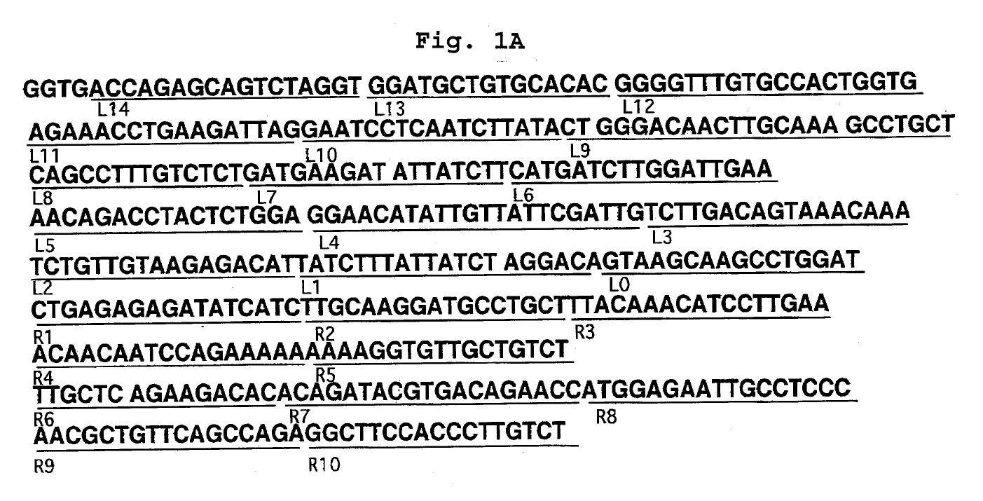

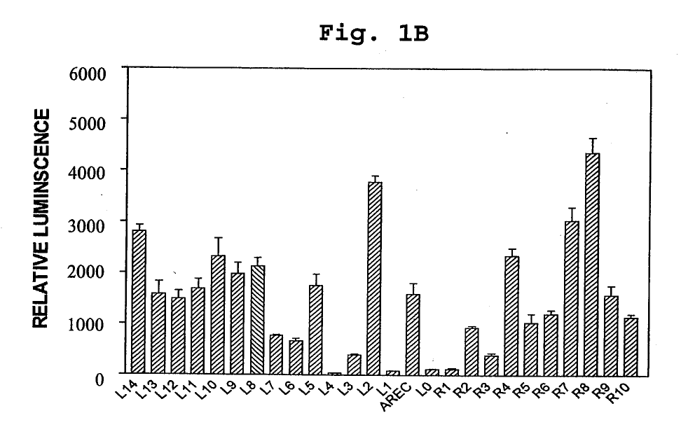

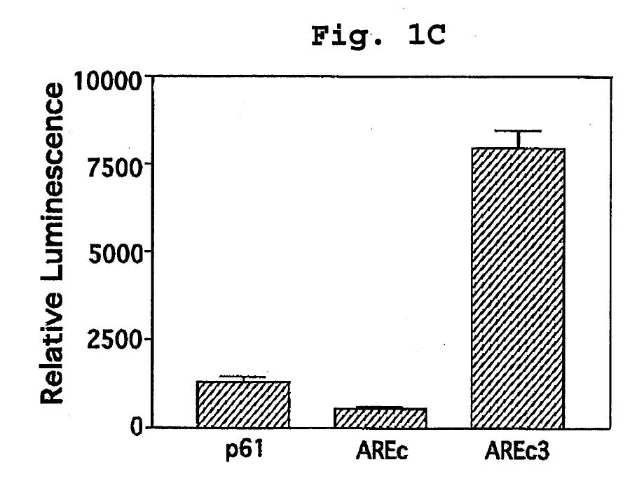

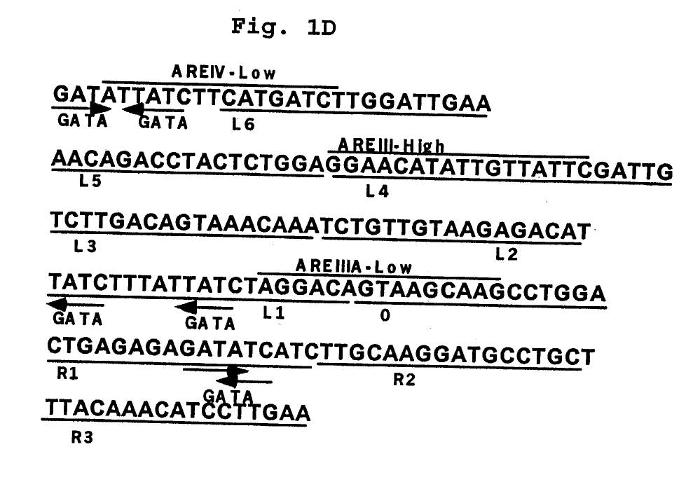

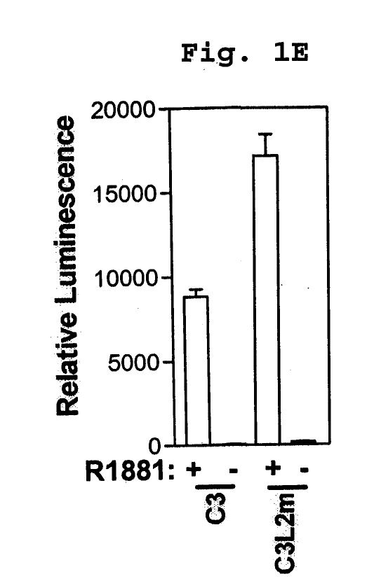

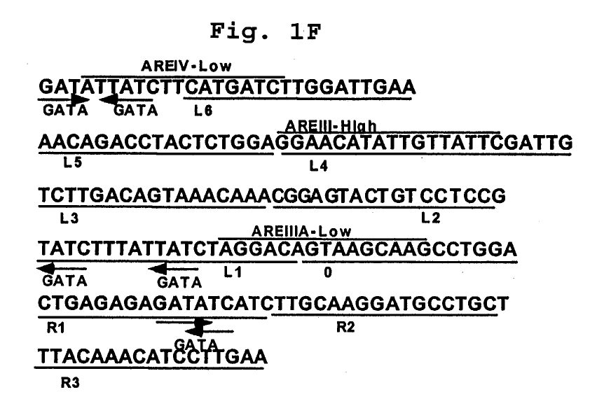

AREc is located 4.2 kb upstream of the PSA promoter and allows the high expression of PSA in a tissue-specific manner (3, 4, 22). Twenty-five linker scanning mutants, wherein a region of 17 nucleotides was replaced with a Gal4 binding sequence, within the AREc region of pGL3/AREc/TATA were constructed to search for important cis-elements (FIG. 1A). LNCaP cells were transfected with 0.5 μg of the constructs and pRL-SV40 (Promega) as a transfection efficiency control. Several linker mutants (L1, L3, L4, L6, L7, R1, R2 and R3) exhibited a clear and significant decrease in transcription activity levels as determined by luciferase expression (FIG. 1B). These results indicate the existence of an activator element located between positions L7 and R3 (AREc3; SEQ ID NO: 14; FIG. 1D). Further analysis of this region reveals that the alteration of the L2 region via PCR-based mutagenesis from the wild-type sequence (TCTGTTGTAAGAGACAT; SEQ ID NO: 16) to a GAL4 binding site sequence (CGGAGTACTGTCCTCCG; SEQ ID NO: 11) enhanced the activity of AREc3 two-fold (FIG. 1E, see AREc3L2m (FIG. 1F; SEQ ID NO: 17)).

To evaluate the promoter activity mediated by AREc3, AREc3 was cloned in the luciferase reporter system and compared with AREc and the 5.8 kb PSA promoter, p61 (22). As shown in FIG. 1C, AREc3 transcription activity levels were 5-fold higher than p61 and 10-fold higher than AREc in the presence of androgen. Similar results were obtained in LNCaP and C4-2 cells. However, all of the constructs had a basal promoter activity level in the absence of androgen (data not shown).

Based on these results, it appeared that AREc3 has high enhancer activity due to the deletion of potential gene silencer elements outside of the AREc3 region. In addition, there are three AR binding sites and three pairs of GATA transcription factor binding sites in AREc3. Mutations in these AR (6; data not shown) and GATA (data not shown) binding sequences decreased AREc3 transcriptional activity. These results demonstrate a role of GATA binding proteins in PSA expression and a collaboration between AR and GATA binding proteins.

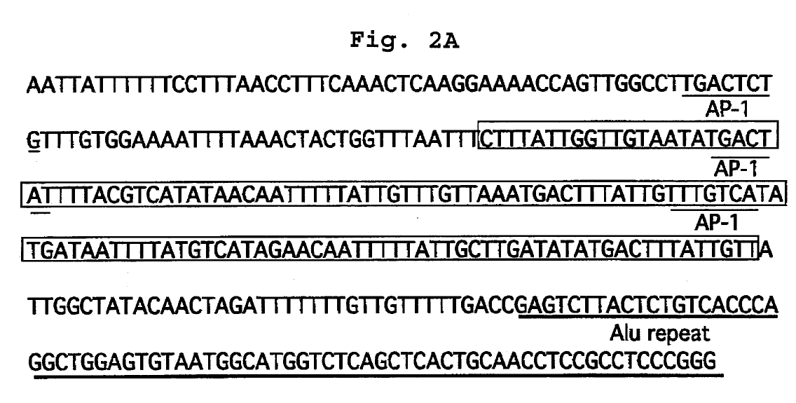

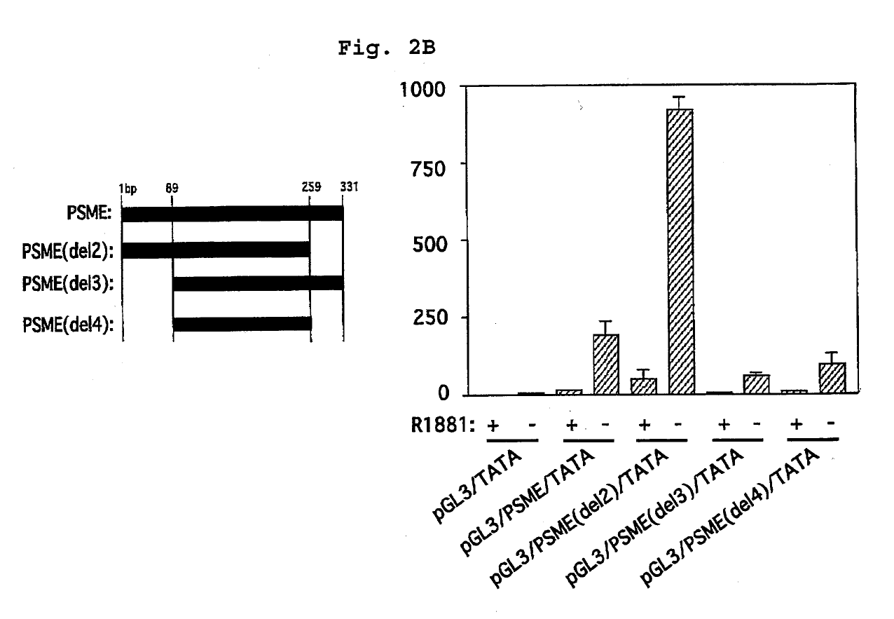

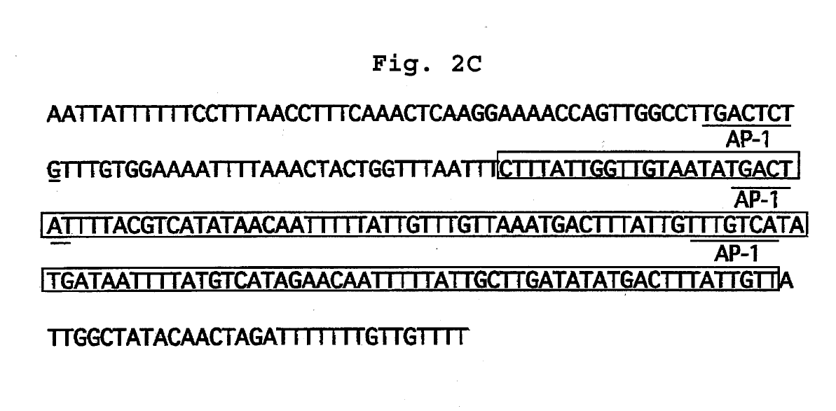

PSME, which is located within the third intron of the PSMA-encoding gene, enables prostate-specific gene expression of PSMA in the absence of androgen (20). Deletion analyses were conducted to further locate prostate-specific enhancer regions within PSME. As shown in FIG. 2B, PSME(del2), which lacked the Alu-repeat sequence (bases 262 to 327) located at the end of the PSME gene, had four to five-fold higher activity than PSME, suggesting that the Alu-repeat contains a suppressive regulatory element. Alu repeats belong to the SINE (Short Interspersed Element) family of human repetitive sequences (24). Although the function of Alu-repeat sequences is not well understood, several studies have identified transcriptional silencers within Alu-repeat sequences and suggested that Alu-repeat sequences might be involved in transcriptional regulation (24). These results provide additional evidence suggesting a role for Alu-repeat sequences in gene regulation.

In contrast to PSME (del2), PSME (del3), which lacks bases 1 to 90, exhibited much lower promoter activity than compared with the full-length PSME, indicating that this region upstream of the direct repeat sequences (FIG. 2A) harbors an enhancer element(s). According to sequence analysis, the upstream region of PSME (from bases 1 to 90) includes an IFN (interferon)-stimulated response element (ISRE), an activator protein-1 (AP-1) binding site and an AP-3 binding site (FIG. 2A). These potential regulators may be involved in the transcriptional activation of PSMA gene expression.

The promoter activity of mutant PSME (del4) was moderately higher than PSME (del3) but lower than PSME (del2), reflecting the overall level of promoter activity without silencing or enhancing regulatory elements in the regions of deleted bases 1 to 90 and 262 to 327, respectively.

All of the PSME deletions in FIG. 2B showed down-regulation in the presence of androgen, suggesting that the regulatory element which is responsible for androgen mediated down-regulation of PSME activity resides in the direct repeat sequence (FIG. 2A).

Interestingly, androgen receptor (AR) was demonstrated to inhibit c-Jun/AP-1 site interaction by forming a complex with c-Jun without directly binding to the AP-1 site (25), suggesting that the decreasing PSMA expression in the presence of androgen is mediated through AP-1. Since there are three AP-1 sites within and upstream of the direct repeat sequence, it is likely that AP-1 plays a role in this androgen effect.

The PSA promoter, which has been investigated for prostate cancer gene therapy (7, 8), is a tissue-specific promoter whose activity heavily depends on androgens. Previous reports demonstrated that the PSA promoter had higher activity in androgen-independent C4-2 cells than androgen-dependent LNCaP cells in the absence of androgen (22). However, its activity in C4-2 cells in the absence of androgen is still much weaker than the commonly used SV40 or RSV promoters. This lower level of promoter activity potentially hampers its application in patients for androgen-ablation therapy. To the contrary, the enhancer activity of PSME (del2) was higher in the absence of androgen, but still significantly lower compared with constitutively active viral promoters (data not shown). It is believed that both AREc3 and PSME (del2) likely functioned weakly in patients under androgen-ablation therapy due to the patients' low levels of androgen and to AR mutation or amplification which resulted in partial activation of AREc3 and suppression of PSME (del2) activity (unpublished data). Thus, it appears that the combination of AREc3 and PSME (del2) has a synergistic enhancer effect by balancing out the positive and negative regulatory effects of androgen and retaining tissue specificity.

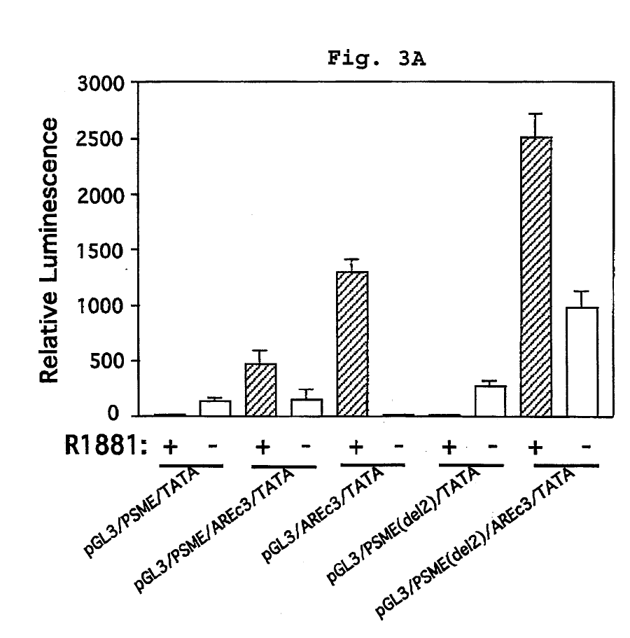

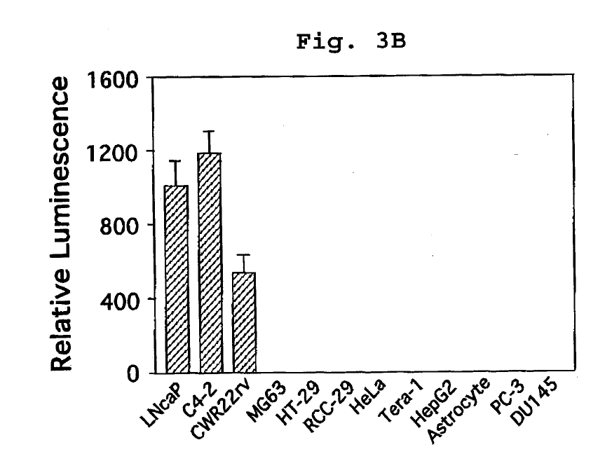

PSES Synergistically Drives Gene Expression in a Prostate-Specific Manner. A chimeric promoter was constructed comprising AREc3 and PSME(del2), called prostate-specific-enhancing-sequence (PSES). Evaluation of PSES's promoter activity is shown in FIG. 3A. The level of luciferase activity driven by PSES was approximately two-fold higher than AREc3 in the presence of androgen and four to five-fold higher than PSME(del2) in the absence of androgen, and approximately two-fold higher than RSV promoter-mediated luciferase activity (data not shown). Notably, promoter activity of PSES was greater than the sum of AREc3 and PSME(del2), suggesting that transcription regulators in AREc3 and PSME(del2) cooperated synergistically to drive a higher level of gene expression. Interestingly, the addition of PSME to AREc3 also elicited a negative effect on the promoter activity of AREc3. Thus, the Alu-repeat appears to interfere with AR-induced transcriptional enhancement of AREc3 by an unknown mechanism.

Although the potency of a promoter is important for gene-therapy purposes, tissue specificity is also critical for avoiding potential non-specific side effects. The tissue specificity of PSES was tested in several different cell lines. As shown in FIG. 3B, PSES drove a high level of luciferase expression in PSA/PSMA-expressing prostate cancer cells: LNCaP, C4-2 and CWR22rv. However, its activity in non-prostate cancer cells and PSMA-negative prostate cancer cells (PC-3 and DU145) was negligible, suggesting that PSES retained tissue specificity similar to the PSA and PSME promoter elements.

PSES Retained Tissue Specificity in an Adenoviral Vector

Adenoviral vectors have been extensively investigated as anti-tumor reagents, demonstrating real promise for prostate cancer gene-therapy (26). To evaluate the potential application of PSES for gene-therapy, a recombinant adenovirus, Ad-PSES-luciferase carrying the luciferase gene under the control of PSES, was constructed as illustrated in FIG. 4A and described hereinabove. PSES was then tested to see if it would exhibit prostate-specific activity in a recombinant adenovirus form. Ad-PSES-luciferase was used to infect several different cell types. As shown in FIG. 4B, PSES was more active in PSA/PSMA-positive prostate cancer cells by 400 to 1000-fold as compared with PSA/PSMA-negative prostate (PC-3 and DU145) or non-prostate cancer cells, demonstrating that PSES retained tissue specificity in a recombinant adenoviral vector.

In order to evaluate the tissue discriminatory promoter activity of PSES in an experimental animal, Balb/c nude mice were injected with 2.0×10⁹ virus particles of Ad-CMV-luciferase or Ad-PSES-luciferase. After 3 days, the luciferase expression in different mouse organs was measured. As shown in FIG. 4C (left panel), high levels of luciferase activity were detected predominantly in liver, spleen and lung, but negligible activity was measured in other tissues obtained from mice injected with Ad-CMV-luciferase. Unlike Ad-CMV-luciferase, Ad-PSES-luciferase was inactive in all organs tested. However, when injected directly into prostate (see right panel in FIG. 4C), Ad-PSES-luciferase was highly active in prostate cells. These data demonstrate that PSES in an adenviral vector was active in normal mouse prostate with high tissue specificity.

Based on these results, PSES may be used to advantage to treat androgen-independent prostate carcinomas. PSES promoter activity was high in PSA/PSMA-positive prostate cancer cells in the presence and absence of androgens and exhibited strong tissue specificity when inserted in an adenoviral vector. Its strong androgen-independent promoter activity makes PSES superior to PSA and PSMA promoters for patients undergoing androgen-ablation therapy.

### REFERENCES

1. Greenlee, R. T., Hill-Harmon, M. B. and Murray, T. Cancer statistics. CA. Cancer J. Clin. 51:15-36, 2001.

2. Cleutjens, K. B., van der Korput, H. A., van Eekelen, C. C., van Rooij, H. C., Faber, P. W., and Trapman, J. Both androgen receptor and glucocorticoid receptor are able to induce prostate-specific antigen expression, but differ in their growth-stimulating properties of LNCaP cells. Mol. Endocrinol. 11: 148-161, 1997.

3. Cleutjens, K. B., van Eekelen, C. C., van der Korput, H. A., Brinkmann, A. O., and Trapman, J. Two androgen response regions cooperate in steroid hormone regulated activity of the prostate-specific antigen promoter. J. Biol. Chem. 271: 6379-6388, 1996.

4. Schuur, E. R., Henderson, G. A., Kmetec, L. A., Miller, J. D., Lamparski, H. G., and Henderson, D. R. Prostate-specific antigen expression is regulated by an upstream enhancer. J. Biol. Chem. 271: 7043-7051, 1996.

5. Zhang, S., Murtha, P. E., and Young, C. Y. Defining a functional androgen responsive element in the 5′ far upstream flanking region of the prostate-specific antigen gene. Biochem. Biophys. Res. Commun. 231:784-788, 1997.

6. Huang, W. et al. Cooperative assembly of androgen receptor into a nucleoprotein complex that regulates the prostate-specific antigen enhancer. J. Biol. Chem., 1999. 274(36): 25756-25768.

7. Gotoh, A., Ko, S. C., Shirakawa, T., Cheon, J., Kao, C., Miyamoto, T., Gardner, T. A., Ho, L. J., Cleutjens, C. B., Trapman, J., Graham, F. L. and Chung, L. W. Development of prostate-specific antigen promoter-based gene therapy for androgen-independent human prostate cancer. J. Urol. 160(1): 220-9, 1998.

8. Wu, L., Matherly, J., Smallwood, A., Adams, J. Y., Billick, E., Belldegrun, A. and Carey, M. Chimeric PSA enhancers exhibit augmented activity in prostate cancer gene therapy vectors. Gene Ther. 8(18): 1416-26, 2001.

9. Ercole, C. J. Prostate specific antigen and prostate acid phosphotase in the monitoring and staging of patienbts with prostate cancer. Journal of Urology, 1987. 138: 1181-1184.

10. Hudson, M. A., Bahnson, R. R., and Catalona, W. J. Clinical use of prostate specific antigen in patients with prostate cancer. Journal of Urology, 1989. 142(4): 1011-1017.

11. Stamey, T. A., et al. Prostate specific antigen in the diagnosis and treatment of adenocarcinoma of the prostate. IV. Anti-androgen treated patients. Journal of Urology, 1989. 141(5): 1088-1090.

12. Carter, R. E., Feldman, A. R. and Coyle, J. T. Prostate-specific membrane antigen is a hydrolase with substrate and pharmacologic characteristics of a neuropeptidase. Proc. Natl. Acad. Sci. U. S. A. 93(2): 749-53, 1996.

13. Pinto, J. T., Suffoletto, B. P., Berzin, T. M., Qiao, C. H., Lin, S., Tong, W. P., May, F., Mukherjee, B. and Heston, W. D. Prostate-specific membrane antigen: a novel folate hydrolase in human prostatic carcinoma cells. Clin Cancer Res. 2(9): 1445-51, 1996.

14. Horoszewicz, J. S., Kawinski, E. and Murphy, G. P. Monoclonal antibodies to a new antigenic marker in epithelial prostatic cells and serum of prostatic cancer patients. Anticancer Res. 7(5B): 927-35, 1987.

15. Xiao, Z., Adam, B. L., Cazares, L. H., Clements, M. A., Davis, J. W., Schellhammer, P. F., Dalmasso, E. A. and Wright, G. L. Jr. Quantitation of serum prostate-specific membrane antigen by a novel protein biochip immunoassay discriminates benign from malignant prostate disease. Cancer Res. 61(16): 6029-33, 2001.

16. Sweat, S.D., et al. Prostate-specific membrane antigen expression is greatest in prostate adenocarcinoma and lymph node metastases. Urology, 1998. 52(4): 637-640.

17. Wright, G. L. Jr., Grob, B. M., Haley, C., Grossman, K., Newhall, K., Petrylak, D., Troyer, J., Konchuba, A., Schellhammer, P. F. and Moriarty, R. Upregulation of prostate-specific membrane antigen after androgen-deprivation therapy. Urology. 48(2): 326-34, 1996.

18. O'Keefe, D. S., Su, S. L., Bacich, D. J., Horiguchi, Y., Luo, Y., Powell, C. T., Zandvliet, D., Russell, P. J., Molloy, P. L., Nowak, N. J., Shows, T. B., Mullins, C., Vonder Haar, R. A., Fair, W. R. and Heston, W. D. Mapping, genomic organization and promoter analysis of the human prostate-specific membrane antigen gene. Biochim. Biophys. Acta. 1443(1-2): 113-27, 1998.

19. Good, D., et al. Cloning and characterization of the prostate-specific membrane antigen promoter. J. Cell. Biochem. 1999. 74(3): 395-405.

20. Watt, F., Martorana, A., Brookes, D. E., Ho, T., Kingsley, E., O'Keefe, D. S., Russell, P. J., Heston, W. D. and Molloy, P. L. A tissue-specific enhancer of the prostate-specific membrane antigen gene, FOLH1. Genomics 73(3): 243-54, 2001.

21. Uchida, A., O'Keefe, D. S., Bacich, D. J., Molloy, P. L. and Heston, W. D. In vivo suicide gene therapy model using a newly discovered prostate-specific membrane antigen promoter/enhancer: a potential alternative approach to androgen deprivation therapy. Urology. 58:2(Suppl 1): 132-9, 2001.

22. Yeung, F., Li, X., Ellett, J., Trapman, J., Kao, C. and Chung, L W. Regions of prostate-specific antigen (PSA) promoter confer androgen-independent expression of PSA in prostate cancer cells. J. Biol. Chem. 275(52): 40846-55, 2000.

23. Graham, F. L. and Prevec, L. Methods for construction of adenovirus vectors. Mol. Biotechnol. 3(3): 207-20, 1995.

24. Sharan, C, Hamilton, N. M., Parl, A. K., Singh, P. K., Chaudhuri, G. Identification and characterization of a transcriptional silencer upstream of the human BRCA2 gene. Biochem. Biophys. Res. Commun. 265(2): 285-90, 1999.

25. Kallio, P. J., Poukka, H., Moilanen, A., Janne, O. A., Palvimo, J. J. Androgen receptor-mediated transcriptional regulation in the absence of direct interaction with a specific DNA element. Mol. Endocrinol. 9(8): 1017-28, 1995.

26. Matsubara, S., Wada, Y., Gardner, T. A., Egawa, M., Park, M. S., Hsieh, C. L., Zhau, H. E., Kao, C., Kamidono, S., Gillenwater, J. Y. and Chung, L. W. A conditional replication-competent adenoviral vector, Ad-OC-Ela, to cotarget prostate cancer and bone stroma in an experimental model of androgen-independent prostate cancer bone metastasis. Cancer Res. 61(16): 6012-9, 2001.

27. Thalmann, G. N., Anezinis, P. E., Chang, S. M., Zhau, H. E., Kim, E. E., Hopwood, V. L., Pathak, S., von Eschenbach, A. C., and Chung, L. W. Androgen-independent cancer progression and bone metastasis in the LNCaP model of human prostate cancer. Cancer Res. 54(10):2577-81, 1994.

28. Navone, N. M., Olive, M., Ozen, M., Davis, R., Troncoso, P., Tu, S. M., Johnston, D., Pollack, A., Pathak, S., von Eschenbach, A. C., and Logothetis, C. J. Establishment of two human prostate cancer cell lines derived from a single bone metastasis. Clin. Cancer Res. 3(12 Pt 1):2493-500, 1997.

29. Sramkoski, R. M., Pretlow, T. G. 2nd, Giaconia, J. M., Pretlow, T. P., Schwartz, S., Sy, M. S., Marengo, S. R., Rhim, J. S., Zhang, D., and Jacobberger, J. W. A new human prostate carcinoma cell line, 22Rv1. In Vitro Cell Dev. Biol. Anim. 35(7):403-9, 1999.

30. Wiley, S. R. et al. Identification and characterization of a new member of the TNF family that induces apoptosis. Immunity. 3(6):673-682, 1995.

31. Hsieh, C. L. et al. A novel targeting modality to enhance adenoviral replication by vitamin D(3) in androgen-independent human prostate cancer cells and tumors. Cancer Res. 62(11):3084-3092, 2002.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A chimeric PSES promoter construct comprising the AREc3 promoter of the PSA gene and the PSME(del2) promoter of the PSMA gene.

2. The PSES promoter construct of claim 1, wherein said AREc3 promoter and said PSME(del2) promoter act synergistically to drive gene expression.

3. The PSES promoter construct of claim 1, wherein said AREc3 promoter is AREc3L2m encoded by the nucleic acid sequence comprising SEQ ID NO: 17.

4. The PSES promoter construct of claim 1, wherein said PSES promoter construct is encoded by the nucleic acid sequence comprising SEQ ID NO: 1.

5. The PSES promoter construct of claim 1, wherein said PSES promoter construct further comprises a heterologous nucleic acid encoding a protein.

6. The PSES promoter construct of claim 5, wherein said heterologous nucleic acid encodes a reporter protein selected from the group consisting of luciferase, β-galactosidase, chloramphenicol acetyltransferase, secreted alkaline phosphatase (SEAP), and green fluorescent protein.

7. The PSES promoter construct of claim 5, wherein said heterologous gene encodes a toxic protein.

8. The PSES promoter construct of claim 7, wherein said toxic protein is selected from the group consisting of cytosine deaminase, thymidine kinase, and a death ligand, TRAIL.

9. The PSES promoter construct of claim 5, wherein said heterologous nucleic acid encodes an angiostatic protein selected from the group consisting of endostatin, tumstatin, sFLK-1 (KDR; kinase insert domain receptor), sFLT-1 (fms-like tyrosine kinase 1 receptor), TSP1 (thrombospondin), and sTie2 (ExTEK).

10. An expression vector comprising the PSES construct of claim 5.

11. The expression vector of claim 10, wherein said heterologous gene encodes a reporter protein.

12. The expression vector of claim 11, wherein said heterologous gene encodes a protein selected from the group consisting of luciferase, β-galactosidase, chloramphenicol acetyltransferase, secreted alkaline phosphatase (SEAP), and green fluorescent protein.

13. The expression vector of claim 10, wherein said heterologous gene encodes a toxic protein selected from the group consisting of cytosine deaminase, thymidine kinase, and the death ligand, TRAIL.

14. The expression vector of claim 10, wherein said heterologous nucleic acid encodes an angiostatic protein selected from the group consisting of endostatin, tumstatin, sFLK-1 (KDR; kinase insert domain receptor), sFLT-1 (fms-like tyrosine kinase 1 receptor), TSP1 (thrombospondin), and sTie2 (ExTEK).

15. The expression vector of claim 10, wherein said expression vector is an adenoviral vector.

16. An isolated host cell transformed with the expression vector of claim 10.

17. The isolated host cell of claim 16, which is selected from the group consisting of a human cell, a tumor cell, a prostate cell, and a prostate tumor cell.

18. The isolated host cell of claim 16, which is an androgen-independent prostate cancer cell.

19. The isolated host cell of claim 16, which is an androgen-dependent prostate cancer cell.

20. The promoter construct of claim 7, wherein said promoter construct is inserted into a vector selected from the group consisting of an adenoviral vector and adeno-associated viral vector, which selectively targets prostate carcinoma cells.

21. The promoter construct of claim 9, wherein said promoter construct is inserted into a vector selected from the group consisting of an adenoviral vector and adeno-associated viral vector, which selectively targets prostate carcinoma cells.

22. The adenoviral vector of claim 20 which is selected from the group consisting of AdE1aPSESE1b, AdTK-PSESE1aPSESE1b, PSESE1aPSESE1b, AdE1aPSESE1b-E4PSES-TK, AdAREc3E1aPSME(del2)E1b, AdAREc3E1aPSME(del2)E1b-E4PSES-TK, AdPSME(del2)E1aAREc3E1b, AdPSME(del2)E1aAREc3E1b-E4PSES-TK, AdTRAIL-PSESE1aPSESE1b, AdE1aPSESE1b-E4PSES-TRAIL, AdAREc3E1aPSME(del2)E1b-E4PSES-TRAIL and AdPSME(del2)E1aAREc3E1b-E4PSES-TRAIL.

23. A method for selectively targeting prostate carcinoma cells for destruction, while sparing normal cells, comprising administering to a patient in need thereof a pharmaceutical composition containing the construct of claim 20.

24. The pharmaceutical composition of claim 23, wherein said pharmaceutical composition comprises an adenoviral vector of claim 22.

25. The method of claim 23, wherein said pharmaceutical composition is administered via direct injection into the prostate.

26. The method of claim 23, wherein said pharmaceutical composition is administered via intravenous injection.

27. A method for identifying a test agent that modulates PSES promoter activity, comprising:
a) providing a reporter construct comprising PSES operably linked to a nucleic acid sequence encoding a reporter protein;
b) identifying a test agent which binds to the PSES promoter; and
c) measuring PSES promoter activity as a function of reporter protein expression levels in the presence and absence of said test agent, a change in the reporter protein expression levels indicating that said test agent modulates PSES promoter activity.

28. The method of claim 27 wherein said PSES promoter construct is introduced into a host cell selected from the group consisting of a human cell, a tumor cell, a prostate cell, and a prostate tumor cell.

29. The method of claim 27, wherein said PSES promoter construct is inserted into an animal thereby generating a transgenic animal.

30. The transgenic animal of claim 27, wherein said animal is a mouse selected from the group consisting of C4-2, CWR22rv and MDA Pca2b.

31. A method for identifying an agent having binding affinity for a PSES promoter construct or fragment thereof, said method comprising:
a) providing a PSES promoter construct or fragment thereof in a biological buffer;
b) contacting said PSES promoter construct or fragment thereof in said biological buffer with a detectably labeled agent suspected of having binding affinity for said PSES promoter construct or fragment thereof, such that a detectably labeled complex forms between those agents having affinity for said PSES promoter construct or fragment thereof; and
d) identifying and isolating said detectably labeled complex if present, thereby identifying said agent.

32. A method as claimed in claim 31, wherein said PSES promoter construct or fragment thereof is adsorbed to a solid support.

33. A method as claimed in claim 32, wherein said method is performed in a high throughput screening format.

34. A method as claimed in claim 31, wherein said PSES promoter construct or fragment thereof is contacted with a plurality of detectably labeled agents present in a chemical combinatorial library.

35. A method for reducing angiogenesis in prostate carcinoma cells, while sparing normal cells, comprising administering to a patient in need thereof a pharmaceutical composition containing the construct of claim 14 in a biologically acceptable carrier.

36. The method of claim 35, wherein said pharmaceutical composition is administered via direct injection into the prostate.

37. The method of claim 35, wherein said pharmaceutical composition is administered via intravenous injection.
